(19) United States
(12) Patent Application Publication
(10) Pub. No.: US 2009/0291440 A1
(43) Pub. Date: Nov. 26, 2009

(54) METHOD FOR SYNTHESIZING NUCLEIC ACID USING DNA POLYMERASE BETA AND SINGLE MOLECULE SEQUENCING METHOD

(76) Inventors: Ken Hirano, Kagawa (JP); Yoshinobu Baba, Kagawa (JP); Mitsuru Ishikawa, Kagawa (JP); Yoshiyuki Mizushina, Kagawa (JP); Takahiro Nishimoto, Kyoto (JP)

Correspondence Address: Cheng Law Group, PLLC, 1100 17th Street, N.W., Suite 503, Washington, DC, 20036, US

(21) Appl. No.: 12/301,766

(22) PCT Filed: Nov. 16, 2006
(86) PCT No.: PCT/JP2006/323377

§ 371 Date: Nov. 20, 2008

(30) Foreign Application Priority Data
May 24, 2006 (JP) 2006-143619

(51) Int. Cl. C12Q 1/68; C12P 19/34

(52) U.S. Cl. 435/6; 435/91.5

(57) ABSTRACT

The present invention provides a nucleic acid synthesis method capable of continuously carrying out an extension reaction and a single molecule sequencing method capable of obtaining base information accurately at high speed. A method for synthesizing a nucleic acid, including the steps of: forming a complex of a target nucleic acid hybridized to a primer oligonucleotide and a DNA polymerase β; allowing the DNA polymerase β to incorporate a fluorescently-labeled dNTP so that the fluorescently-labeled dNTP is bound to the 3′ end of the primer oligonucleotide; and allowing the DNA polymerase β to continuously incorporate fluorescently-labeled dNTP to extend a nucleic acid complementary to the target nucleic acid from the 3′ end of the bound fluorescently-labeled dNTP. A method for sequencing a single nucleic acid molecule, including the steps of said method for synthesizing a nucleic acid, wherein fluorescence emitted from each of the fluorescently-labeled dNTP incorporated into the DNA polymerase β is sequentially detected to carry out the sequencing of the target nucleic acid.

### TECHNICAL FIELD

The present invention relates to a technique for synthesizing a nucleic acid. Further, the present invention relates to a technique for analyzing the nucleic acid of an organism. Particularly, the present invention relates to a technique for analyzing a single nucleic acid molecule. More specifically, the present invention relates to a method for synthesizing a nucleic acid and single molecule sequencing using DNA polymerase β.

### BACKGROUND ART

As a typical example of a conventional DNA sequencing method, there is the so-called Sanger method using four-color fluorescence and electrophoresis. Further, as a method for analyzing a single DNA molecule, there is a method using Klenow fragment (DNA polymerase), which has been carried out by Quake et al. and is described in U.S. Pat. No. 6,818,395 and Proceeding of the National Academy of Science of the United States of America, vol. 100, p.p. 3960-3964 (2003).

Patent Document 1: U.S. Pat. No. 6,818,395

Non-Patent Document 1: Proceeding of the National Academy of Science of the United States of America, vol. 100, p.p. 3960-3964 (2003)

### DISCLOSURE OF THE INVENTION

### Object of the Invention

The Sanger method involves the following problems: pre-treatment is complicated; the number of bases that can be read at one time is at most about 800; it takes several hours to analyze one sample; etc.

The method by Quake et al. involves the following problems.

First, it takes much time to analyze a template DNA because one kind of base on the template DNA is analyzed by preparing four kinds of deoxyribonucleotides having different fluorescent labels, and orderly introducing the four kinds of solutions therein to determine the occurrence or nonoccurrence of base incorporation according to the deoxyribonucleotide.

Further, this method uses Klenow fragment, but this enzyme cannot continuously incorporate several fluorescently-labeled deoxyribonucleotides or more, and therefore a base length analyzable by this method is limited to several bases or less. For this reason, this method is not suitable for practical use.

Further, Klenow fragment has a little 3′→5′ exonuclease activity, and therefore removes a synthesized base and then restarts synthesis. For this reason, it is not possible to obtain accurate base information.

It is therefore an object of the present invention to provide a nucleic acid synthesis method capable of continuously carrying out an extension reaction and a single molecule sequencing method capable of obtaining base information accurately at high speed.

### SUMMARY OF THE INVENTION

The present inventors have found that the above object of the present invention can be achieved by using DNA polymerase β as a nucleic acid polymerizing enzyme, and this finding has led to the completion of the present invention.

The present invention includes the following inventions (1) to (8).

The invention disclosed as the following (1) is directed to a method for synthesizing a nucleic acid using fluorescently-labeled deoxyribonucleotides as substrates and DNA polymerase β as a nucleic acid polymerizing enzyme.

(1) A method for synthesizing a nucleic acid, including the steps of:

forming a complex of a target nucleic acid hybridized to a primer oligonucleotide and a DNA polymerase β;

allowing the DNA polymerase β to incorporate a fluorescently-labeled deoxyribonucleotide so that the fluorescently-labeled deoxyribonucleotide is bound to the 3′ end of the primer oligonucleotide; and

allowing the DNA polymerase β to continuously incorporate fluorescently-labeled deoxyribonucleotides to extend a nucleic acid complementary to the target nucleic acid from the 3′ end of the bound fluorescently-labeled deoxyribonucleotide.

(2) The nucleic acid synthesis method according to the above (1), wherein the fluorescently-labeled deoxyribonucleotide is a deoxyribonucleotide labeled with an anionic fluorescent dye.

(3) The nucleic acid synthesis method according to the above (2), wherein the anionic fluorescent dye is selected from a group consisting of Alexa Fluor®488, Alexa Fluor®532, Alexa Fluor®546, fluorescein, Oregon Green®488, Cy3.5, Cy5, Cy5.5, and naphthofluorescein.

The invention disclosed as the following (4) is directed to a method for carrying out single molecule sequencing by detecting fluorescence emitted from each fluorescently-labeled deoxyribonucleotide incorporated into DNA polymerase β by the use of the method for synthesizing a nucleic acid using fluorescently-labeled deoxyribonucleotides as substrates and DNA polymerase β as a nucleic acid polymerizing enzyme.

(4) A method for sequencing a single nucleic acid molecule, including the steps of:

forming a complex of a target nucleic acid to be sequenced hybridized to a primer oligonucleotide and a DNA polymerase β;

allowing the DNA polymerase β to incorporate a fluorescently-labeled deoxyribonucleotide so that the fluorescently-labeled deoxyribonucleotide is bound to the 3′ end of the primer oligonucleotide; and

allowing the DNA polymerase β to continuously incorporate fluorescently-labeled deoxyribonucleotides to extend a nucleic acid complementary to the target nucleic acid to be sequenced from the 3′ end of the bound fluorescently-labeled deoxyribonucleotide, wherein

fluorescence emitted from each of the fluorescently-labeled deoxyribonucleotides incorporated into the DNA polymerase β is sequentially detected to carry out the sequencing of the target nucleic acid.

(5) The method for sequencing a single nucleic acid molecule according to the above (4), wherein two or more kinds of the fluorescently-labeled deoxyribonucleotides are prepared and the two or more kinds of fluorescently-labeled deoxyribonucleotides have different fluorescent labels depending on the kind of their base.

More specifically, the two or more kinds of fluorescently-labeled deoxyribonucleotides are fluorescently labeled forms of at least two kinds of deoxyribonucleotides selected from dATP, dUTP, dTTP, dCTP, and dGTP, and are designed to have different fluorescent labels depending on the kind of their base.

Further, the invention disclosed as the following (6) is also directed to a high-speed single molecule sequencing method using total internal reflection fluorescence microscopy technology.

(6) The method for sequencing a single nucleic acid molecule according to the above (4) or (5), wherein either the target nucleic acid to be sequenced or the DNA polymerase β is immobilized onto a substrate, and wherein an evanescent field is generated at the surface of the substrate, onto which the target nucleic acid to be sequenced or the DNA polymerase β has been immobilized, and wherein when the fluorescently-labeled deoxyribonucleotide is incorporated into the DNA polymerase β, fluorescence emitted from the incorporated fluorescently-labeled deoxyribonucleotide and excited by the evanescent field is detected.

(7) The method for sequencing a single nucleic acid molecule according to any one of the above (4) to (6), wherein the fluorescently-labeled deoxyribonucleotide is a deoxyribonucleotide labeled with an anionic fluorescent dye.

(8) The method for sequencing a single nucleic acid molecule according to the above (7), wherein the anionic fluorescent dye is selected from a group consisting of Alexa Fluor®488, Alexa Fluor®532, Alexa Fluor®546, fluorescein, Oregon Green®488, Cy3.5, Cy5, Cy5.5, and naphthofluorescein.

According to the present invention, it is possible to provide a nucleic acid synthesis method capable of continuously carrying out an extension reaction and a single molecule sequencing method capable of accurately obtaining base information at high speed.

### BRIEF DESCRIPTION OF THE DRAWINGS

FIG. 1 is an illustration schematically showing a single molecule sequencing method according to the present invention using TIRFM.

FIG. 2 shows the result of Example 1 carried out for determining the activity of incorporation of fluorescently-labeled deoxyribonucleotides into DNA polymerase β used as a nucleic acid polymerizing enzyme.

FIG. 3 shows the result of Comparative Example 1 carried out for determining the activity of incorporation of fluorescently-labeled deoxyribonucleotides into Klenow fragment used as a nucleic acid polymerizing enzyme.

FIG. 4 shows the result of Comparative Example 2 carried out for determining the activity of incorporation of fluorescently-labeled deoxyribonucleotides into Sequenase used as a nucleic acid polymerizing enzyme.

FIG. 5 is an illustration showing an optical system used in Example 2.

FIG. 6 shows a result indicating that a single fluorescent molecule (Coumarine) was detected in real time in Example 2.

FIG. 7 shows a result indicating that a single fluorescent molecule (Alexa 488) was detected in real time in Example 2.

FIG. 8 shows a result indicating that a single fluorescent molecule (Cy3.5) was detected in real time in Example 2.

FIG. 9 shows a result indicating that a single fluorescent molecule (Cy5) was detected in real time in Example 2.

FIG. 10 is an illustration schematically showing a single molecule sequencing method carried out in Example 3.

FIG. 11 shows a result indicating that single molecule sequencing was achieved in Example 3.

### MODES FOR CARRYING OUT THE INVENTION

### [Nucleic Acid Synthesis Method]

A nucleic acid synthesis method according to the present invention uses fluorescently-labeled deoxyribonucleotides as substrates and polymerase β as a nucleic acid polymerizing enzyme. A target nucleic acid as a template and an oligonucleotide as a primer are not particularly limited. These components are subjected to conventional nucleic acid synthesis reaction conditions. As a result, a complex of the target nucleic acid hybridized to the primer oligonucleotide and the DNA polymerase β is formed, and then a fluorescently-labeled deoxyribonucleotide is incorporated into the DNA polymerase β and bound to the 3′ end of the primer oligonucleotide.

It has been already found by inventors that DNA polymerase β to be used as a nucleic acid polymerizing enzyme in the present invention shows a very high fluorescently-labeled deoxyribonucleotide incorporation activity. Unlike, For example, Klenow fragment conventionally used as a nucleic acid polymerizing enzyme, DNA polymerase β can continuously incorporate fluorescently-labeled deoxyribonucleotides and synthesize a nucleic acid without stopping the incorporation of several bases.

Further, many nucleic acid polymerizing enzymes have a 3′→5′ exonuclease activity serving as a proofreading function, i.e., the function of removing a base synthesized by the nucleic acid polymerizing enzyme itself. However, DNA polymerase β to be used as a nucleic acid polymerizing enzyme in the present invention is conventionally known as a repair enzyme and does not have such a 3′→5′ exonuclease activity. This makes it possible to stably extend a nucleic acid complementary to a target nucleic acid from the 3′ end of a fluorescently-labeled deoxyribonucleotide initially bound to the target nucleic acid.

The kind of fluorescent label, i.e., fluorescent functional group is not particularly limited. From the viewpoint of the activity of incorporation into DNA polymerase β, for example, anionic fluorescent dyes are preferably used. Examples of the anionic fluorescent dyes include Alexa Fluor 488, 532, and 546, fluorescein, Oregon Green 488, Cy3.5, 5, and 5.5, and Naphthofluorescein.

An example of application embodiment of the nucleic acid synthesis method according to the present invention is as follows. Either a primer oligonucleotide or a target nucleic acid may be immobilized onto a substrate or the like. This makes it possible to extend and immobilize long fluorescently-modified DNA which has not been able to be stably extended and immobilized.

Another example of application embodiment of the nucleic acid synthesis method according to the present invention is as follows. The nucleic acid synthesis method may be applied to a conventional nucleic acid replication system, and a replication initiation site or a replication initiation sequence may be determined by carrying out fluorescence detection. That is, mapping of a replication origin can be carried out. In the case of carrying out fluorescence detection, it is preferred that the fluorescent labels of deoxyribonucleotides are selected so as to emit fluorescence with different wavelengths depending on the kind of deoxyribonucleotide used. In this case, sequencing can be carried out by detecting incorporated fluorescent labels, which will be described in detail later with reference to a single molecule sequencing method.

### <Single Molecule Sequencing Method>

In a single molecule sequencing method according to the present invention, fluorescently-labeled deoxyribonucleotides are used as substrates and DNA polymerase β is used as a nucleic acid polymerizing enzyme to carry out nucleic acid synthesis in the same manner as the nucleic acid synthesis method described above. Then, sequencing of a target nucleic acid as a template is carried out by detecting fluorescence emitted from incorporated fluorescently-labeled deoxyribonucleotide. It is to be noted that the fluorescent labels of deoxyribonucleotides are selected so as to emit fluorescence with different wavelengths depending on the kind of deoxyribonucleotide used.

A means for fluorescence detection is not particularly limited, but a means capable of carrying out detection with single base resolution is preferably used.

Examples of a means capable of carrying out detection with single base resolution include a method described in U.S. Pat. No. 6,818,395 and Proceeding of the National Academy of Science of the United States of America, 100, 3960-3964 (2003). According to such a method, necessary kinds (usually, four kinds) of fluorescently-labeled deoxyribonucleotides are prepared, and then each solutions of the necessary kinds of fluorescently-labeled deoxyribonucleotides is orderly introduced and then washed out, and this process is repeated to carry out analysis while determining the occurrence or nonoccurrence of base incorporation for each kind of fluorescently-labeled deoxyribonucleotide.

Another example of a means capable of carrying out detection with single base resolution is a method using total internal reflection fluorescence microscopy (TIRFM). In this case, either a target nucleic acid to be sequenced or DNA polymerase β is immobilized onto a substrate, and an evanescent field is generated at the surface of the substrate where a target nucleic acid to be sequenced or DNA polymerase β has been immobilized. When a fluorescently-labeled deoxyribonucleotide is incorporated into DNA polymerase β in a nucleic acid synthesis reaction, a fluorescent label of the incorporated fluorescently-labeled deoxyribonucleotide is excited by the evanescent field, and fluorescence emitted by excitation is detected.

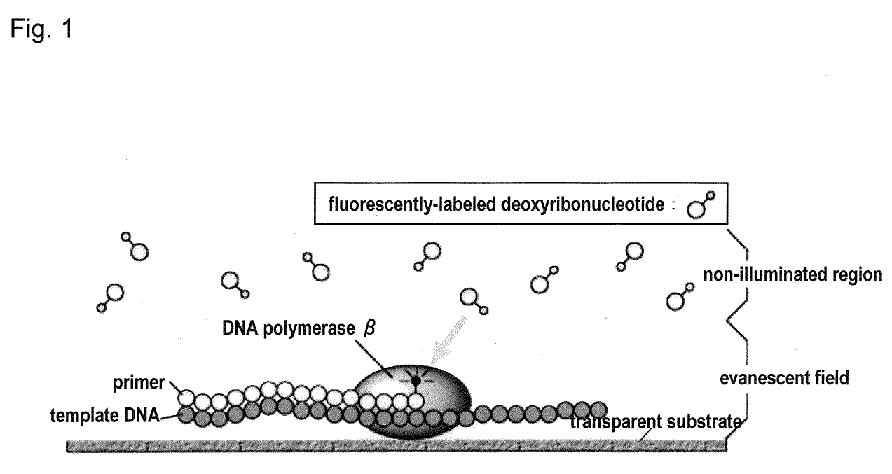

FIG. 1 is a schematic view showing a specific example of the method using TIRFM. In FIG. 1, DNA polymerase β is immobilized onto a substrate (transparent substrate) to carry out replication of a target nucleic acid (template DNA) as a template with the use of a primer. Alternatively, the target nucleic acid may be immobilized onto the substrate instead of the DNA polymerase β. Then, necessary kinds of deoxyribonucleotides are prepared and modified so as to emit fluorescence with different wavelengths depending on the kind of deoxyribonucleotide. These deoxyribonucleotides are used as fluorescently-labeled deoxyribonucleotides.

In order to excite fluorescent molecules, total internal reflection illumination is used to generate an evanescent field at the surface of the substrate. Evanescent light exudes within a limited region extending up to about 200 nm above the surface of the substrate, and therefore a region outside the limited region is a non-illuminated region. This makes it possible to observe a fluorescence phenomenon occurring in the limited region with high sensitivity under conditions where there is little background fluorescence.

The fluorescently-labeled deoxyribonucleotides undergo rapid Brownian motion which cannot be caught by a detection camera, and therefore the fluorescently-labeled deoxyribonucleotides present in an illuminated region cannot be usually recognized. However, when the fluorescently-labeled deoxyribonucleotides are incorporated into the DNA polymerase β, they undergo restricted Brownian motion and therefore it becomes possible to recognize them by a detection camera. This makes it possible to differentiate between incorporated deoxyribonucleotides and unincorporated deoxyribonucleotides, i.e., free deoxyribonucleotides floated in a solution.

Further, fluorescence emitted from an excited fluorescent molecule disappears due to the action of an active enzyme generated by excitation light by the time when a fluorescent molecule subsequently incorporated and emits fluorescence or before the fluorescence emitted from the subsequently-incorporated fluorescent molecule disappears. Therefore, it is possible to detect only a desired fluorescent molecule. The sequencing of a target nucleic acid is carried out by reading the wavelength and/or intensity of fluorescence emitted from fluorescent molecules sequentially. Sequencing using TIRFM is carried out by observing an enzyme reaction for nucleic acid synthesis on a real-time scale, and therefore high-speed sequencing can be achieved. For example, the speed of the sequencing is about 10 to 50 bases per second.

As a substrate for immobilizing the fluorescently-labeled deoxyribonucleotides or DNA polymerase β, one which has a higher index of refraction than a reaction solution for use in the extension of a nucleic acid and which allows an evanescent field to be generated on the reaction solution side when laser light undergoes total internal reflection at the interface between the substrate and the reaction solution is used. The substrate is made of a material through which at least light can pass. For example, a substrate made of a material having a high light transmittance, such as a glass substrate or a substrate made of a resin (e.g., polycarbonate, PMMA), is preferably used.

A method for immobilization onto a substrate is not particularly limited, and may be appropriately selected by those skilled in the art. More specifically, immobilization is appropriately carried out by, for example, utilizing binding between avidin and biotin, binding between digoxigenin and digoxigenin antibody, or binding formed between functional groups using a linker reagent, such as covalent binding between an amino group and a carboxyl group via EDC (1-Ethyl-3-[3-dimethylaminopropyl]carbodiimide Hydrochloride) or NHS (N-hydroxysuccinimide). In the case of immobilizing a target nucleic acid, it is preferably immobilized via a primer sequence of about 10 to 20 bp.

In the case of immobilizing DNA polymerase β, it is preferably immobilized without losing its activity. This is preferably achieved by expressing DNA polymerase β as an affinity tag fusion protein. Examples of an affinity tag include GST (Glutathione S-transferase), 6×His (histidine), and avidin. In a case where GST, 6×His, or avidin is used, immobilization is carried out by utilizing binding between GST and anti-GST, binding between 6×His and 6×His antibody or Ni-NTA (Nitrilotriacetic acid) or binding between avidin and biotin, respectively.

### EXAMPLES

Hereinbelow, the present invention will be described in more detail with reference to the following examples, but is not limited by these examples.

<1. Comparison of Activity of Incorporation of Fluorescently-Labeled Deoxyribonucleotides into Nucleic Acid Polymerizing Enzyme>

In the following Example 1 and Comparative Examples 1 and 2, a comparison of activity of incorporation into a nucleic acid polymerizing enzyme was made among various fluorescent molecule-labeled deoxyribonucleotides.

### Example 1

In Example 1, the activity of incorporation of 22 kinds of dUTPs labeled with different fluorescent molecules as substrates was determined by sequence gel analysis with the use of a nucleic acid composed of a primer sequence and adenines (5′-AAAAA AAAAA CCCTC ACGCT GCCAT CCTCC-3′; SEQ ID No. 1) as a template DNA, a digoxigenin-labeled primer oligonucleotide (5′DIG-GGAGG ATGGC AGCGT GAGGG-3′; SEQ ID No. 2), and calf-derived DNA polymerase β as a nucleic acid polymerizing enzyme. The 22 kinds of fluorescent labels used for labeling dUTPs are as follows.

CascadeBlue

Coumarine

Alexa Fluor 488

Dimethylcoumarine

BODIPY FL

Fluorescein

Fluorescein Chlorotriazinyl

OregonGreen 488

Rohdamine Green

Alexa Fluor 532

Alexa Fluor 546

Alexa Fluor 594

BODIPY TMR

Cy3

Lissamine Rohdamine B

Tetramethylrohdamine

Texas Red

BODIPY 630/650

Cy5

Cy5.5

Naptofluorescein

Cy3.5

Sequence gel analysis was carried out in the following manner. First, the template DNA and the primer oligonucleotide were mixed and left standing at room temperature for 5 minutes to carry out annealing. A mixed solution (10 μL) comprising the template DNA annealed to the primer oligonucleotide, the DNA polymerase β, and the fluorescently-labeled dUTP in a reaction buffer (final concentrations: 0.1 μM primer oligonucleotide/template DNA, 10 μM DNA polymerase β, 10 μM fluorescently-labeled dUTP, 50 mM Tris-HCl (pH 8.0), 1 mM DTT, 5 mM magnesium chloride, 15 v/v % glycerol) was subjected to reaction at 37° C. for 5 minutes. After the completion of the reaction, 7 μL of a reaction quenching solution (95 v/v % formamide, 20 mM EDTA (pH 7.5), 0.1 w/v % XylenCyanolFF, 0.1 w/v % BromophenolBlue) was added to the mixed solution, and a resultant mixture was heated at 95° C. for 5 minutes and then rapidly cooled on ice.

Electrophoresis using a sequence gel plate was performed to analyze the primer oligonucleotide extended by the reaction. 3 μL of the solutions obtained by carrying out reaction and quenching the reaction in such a manner as described above was placed in a well of a sequence gel (composition: 8 w/v % polyacrylamide, 12 M urea, 1×TBE) and electrophoresed for 3 hours. After the completion of the electrophoresis, a nylon membrane was directly placed on the gel and left standing for 30 minutes to transfer the electrophoresed primer oligonucleotides from the gel to the nylon membrane (contact blotting). The nylon membrane, to which the primer oligonucleotides had been transferred, was irradiated with UV at 200 mJ/cm² for 1 minute in a UV cross linker to immobilize the primer oligonucleotides to the nylon membrane.

In order to detect the primer oligonucleotides by chemiluminescence, the following operation was carried out (always at room temperature). The nylon membrane, to which the primer oligonucleotides had been immobilized, was shaken for 1 minute in a washing buffer (0.1 M maleic acid, 0.15 M NaCl, 0.3 v/v % Tween 20, pH 7.5), and was then shaken for 30 minutes in a blocking solution (0.1 M maleic acid, pH 7.5, 10% (w/v) blocking reagent manufactured by Roche Diagnostics K.K. (product number: 1096176)), and was then shaken for 1 hour in a solution obtained by diluting an alkaline phosphatase-labeled digoxigenin antibody solution (concentration: 0.75 U/μL) in 1000 fold with the blocking solution.

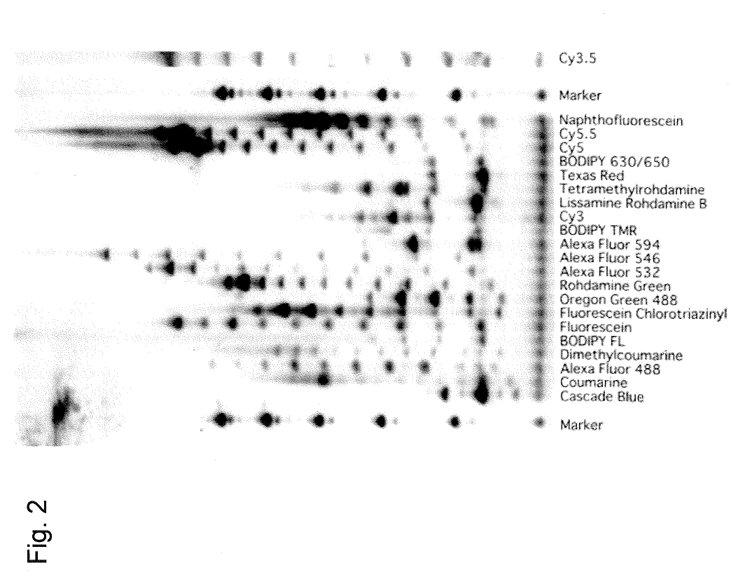

Then, the nylon membrane was shaken for 10 minutes in the washing buffer, and this washing process using the washing buffer was repeated three times. Then, the nylon membrane was shaken for 2 minutes in a detection buffer (0.1 M Tris-HCl, 0.1 M NaCl, pH 9.5). Then, a solution obtained by diluting a CDP-Star solution (concentration: 25 mM) as a chemiluminescence substrate in 1000 fold with the detection buffer was dropped onto the entire nylon membrane subjected to the above operation and left standing for 15 minutes. Then, an X-ray film was exposed to the nylon membrane to obtain an image and then developed. The result is shown in FIG. 2.

### Comparative Example 1

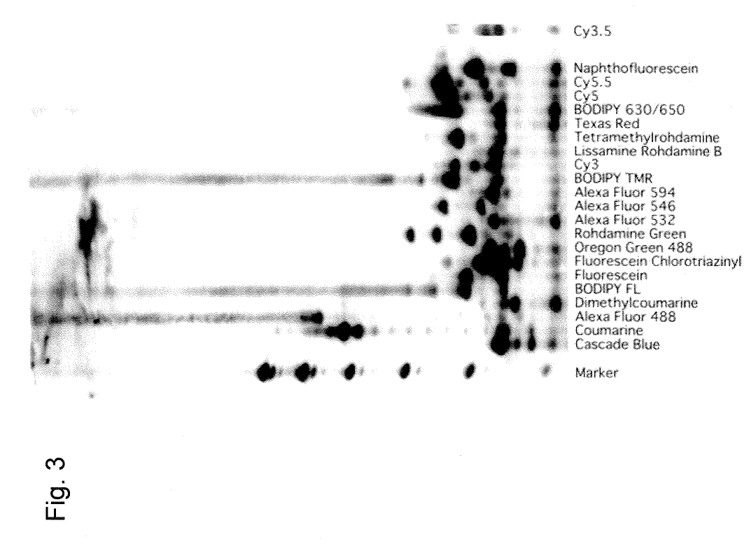

Sequence gel analysis was carried out in the same manner as in Example 1 except that the nucleic acid polymerizing enzyme was changed to Klenow fragment and the composition of the polymerization reaction solution was changed (final concentrations: 0.1 μM primer oligonucleotide/template DNA, 2 U Klenow fragment, 10 μM fluorescently-labeled dUTP, 50 mM Tris-HCl (pH 7.5), 0.1 mM DTT, 7 mM magnesium chloride). The result is shown in FIG. 3.

### Comparative Example 2

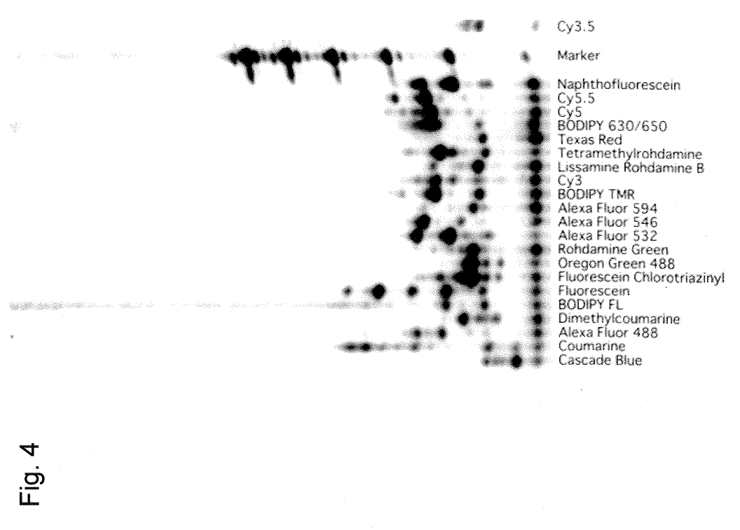

Sequence gel analysis was carried out in the same manner as in Example 1 except that the nucleic acid polymerizing enzyme was changed to Sequenase Version 2.0 manufactured by Amersham (i.e., a genetically-engineered enzyme obtained by depriving T7 DNA polymerase of its 3′→5′ nuclease function) and the composition of the polymerization reaction solution was changed (final concentrations: 0.1 μM primer oligonucleotide/template DNA, 2 U Sequenase Version 2.0, 10 μM fluorescently-labeled dUTP, 40 mM Tris-HCl (pH 7.5), 50 mM NaCl, 20 mM magnesium chloride). The result is shown in FIG. 4.

### <Evaluation of Comparison of Activity of Incorporation>

The result of Example 1 using DNA polymerase β as a nucleic acid polymerizing enzyme indicates that the polymerase β could continuously incorporate fluorescently-labeled dUTP. Further, as can be seen from FIG. 2, the activity of incorporation of dUTPs having anionic fluorescent labels is higher than that of incorporation of dUTPs having highly hydrophobic fluorescent labels, such as BODIPY® series, or cationic or neutral fluorescent labels.

On the other hand, in the case of Comparative Example 1 using Klenow fragment as a nucleic acid polymerizing enzyme, full-length DNA could not be synthesized except for a case where Coumarine-labeled dUTP was used and a case where Alexa Fluor 488-labeled dUTP was used. In the case of Comparative Example 2 using Sequenase as a nucleic acid polymerizing enzyme, full-length DNA could not be synthesized and the synthesis reaction completely stopped after at most 5 bases were incorporated, except for a case where Coumarine-labeled dUTP was used.

These results demonstrate that DNA polymerase β can continuously incorporate fluorescent molecule-labeled deoxyribonucleotides.

### <2. Real-Time Fluorescence Detection>

### Example 2

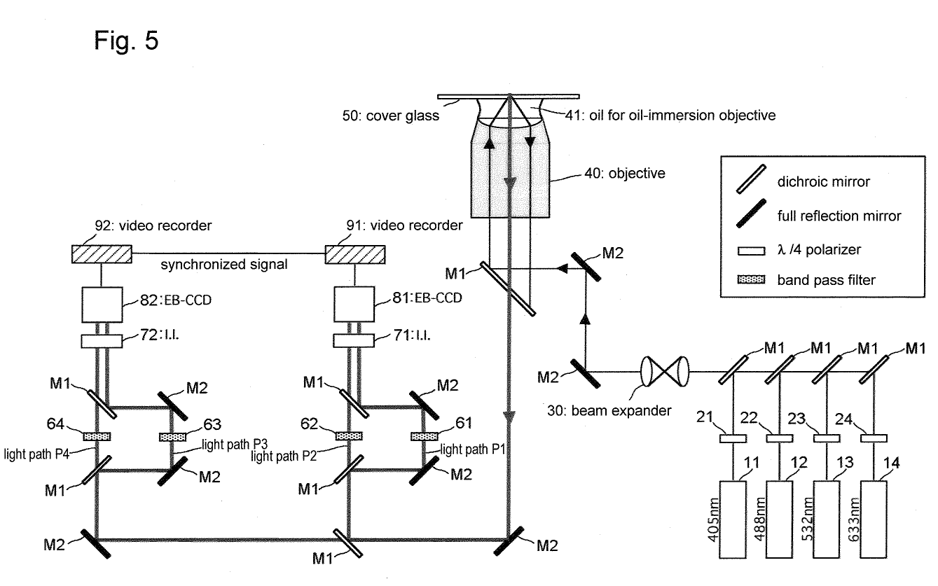

Real-time detection of single fluorescent molecules was carried out using four kinds of fluorescent dyes which had been efficiently incorporated into DNA polymerase β in Example 1 (i.e., Coumarine (excitation: 402 nm, fluorescence: 443 nm), Alexa 488 (excitation: 495 nm, fluorescence: 519 nm), Cy3.5 (excitation: 550 nm, fluorescence 570 nm), and Cy5 (excitation: 650 nm, fluorescence: 667 nm)). An optical system used for the real-time fluorescence detection is shown in FIG. 5. In this optical system, an objective-type total internal reflection fluorescence microscope (inverted type) was used as one example of total internal reflection fluorescence microscopes.

As shown in FIG. 5, in this optical system, four kinds of laser sources (11), (12), (13), and (14) with wavelengths of 405 nm, 488 nm, 532 nm, and 633 nm were installed to excite the four kinds of fluorescent dyes respectively. Laser beams emitted from the laser sources (11), (12), (13), and (14) were converted into circular polarized light by λ/4 polarizers (21), (22), (23), and (24), respectively. The converted laser beams was subjected to optical adjustment so that the four kinds of laser beams were coaxially aligned using a dichroic mirror (M1).

The beam diameter of each of the laser beams guided to a coaxial light path was adjusted by a beam expander (30) provided to adjust a region illuminated with an evanescent field at the surface of a cover glass (50) as a transparent substrate. Then, each of the laser beams whose beam diameter had been adjusted was reflected off appropriately-arranged full reflection mirrors (M2) and dichroic mirrors (M1) so that its optical path was changed, and was then incident on an objective (40) of the inverted-type microscope. The space between the objective (40) and the cover glass (50) was filled with oil (41) for oil-immersion objectives so that the objective was immersed in the oil. The laser beams was subjected to optical adjustment so that the laser beams were refracted by the objective (40) and then allowed to incident on the cover glass at an angle larger than a critical angle (61.0°) (i.e., so that a total internal reflection phenomenon occurred), thereby allowing a sample placed on the cover glass (50) to be irradiated with evanescent light.

In the case of an objective-type total internal reflection fluorescence microscope, the use of an objective having a numerical aperture larger than 1.4 makes it possible to allow laser light to incident on a cover glass at an angle larger than a critical angle (61.0°) determined by the index of refraction of glass (1.52) and the index of refraction of water (1.33) and thereby to allow a total internal reflection phenomenon to occur. Therefore, in the optical system shown in FIG. 5, a 150× objective having a numerical aperture of 1.45 was used as an objective (40) to generate an evanescent field.

A fluorescent dye sample solution was dissolved in a TE buffer (10 mM Tris-HCl, 1 mM EDTA, pH 7.4) so that its final concentration became 1 nM. 10 μL of the thus obtained sample solution was placed on the cover glass (50) having a thickness of 0.12 to 0.17 mm, and was then covered with the same cover glass and observed. No coating was applied onto the surface of the cover glass (50), and single fluorescent dye molecules non-specifically adsorbed to the surface were observed.

The fluorescent images of single fluorescent dye molecules obtained by allowing a total internal reflection phenomenon to occur were classified into four kinds according to their wavelength by dichroic mirrors (M1) appropriately arranged together with full reflection mirrors (M2). In the optical system shown in FIG. 5, dichroic mirrors (M1) suitable for the above-described fluorescence wavelengths of the fluorescent dyes respectively were used so that fluorescence emitted from the fluorescent dyes of a light path P1 (Coumarine), a light path P2 (Alexa 488), a light path P3 (Cy3.5), and a light path P4 (Cy5) could be observed. Similarly, band pass filters suitable for the above-described fluorescence wavelengths of the fluorescent dyes respectively were used as band pass filters (61), (62), (63), and (64).

The single molecule fluorescence images classified according to their wavelength emitted very weak fluorescence, and therefore the image intensifiers (abbreviated as “I.I.”) (71) and (72) were inserted and the light intensity was amplified by so as to be able to be detected by EB-CCD cameras (81) and (82). Single molecule fluorescence images captured by the two EB-CCD cameras (81) and (82) were recorded by video recorders (91) and (92) in digital video (DV) format on video frames (at 30 frames/sec). The two video recorders (91) and (92) were synchronized to allow them to maintain the same timing in recording.

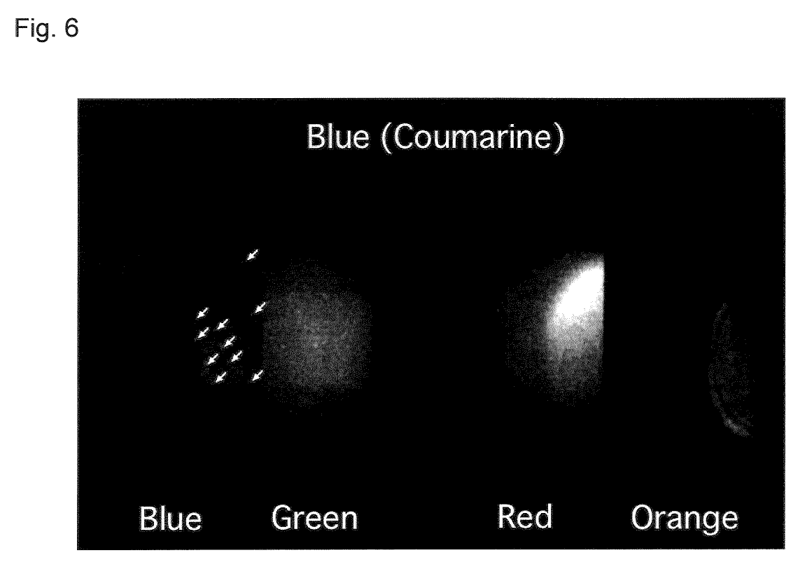

FIGS. 6 to 9 show the result of real-time single molecule detection of the four kinds of fluorescent dyes. The four kinds of dyes (i.e., Coumarine, Alexa 488, Cy3.5, and Cy5) were observed singly by allowing four kinds of laser beams (wavelength: 405 nm, 488 nm, 532 nm, and 633 nm) to incident on the objective at the same time to confirm that only the single fluorescent dye molecules emitting fluorescence having a desired wavelength could be observed by the optical system.

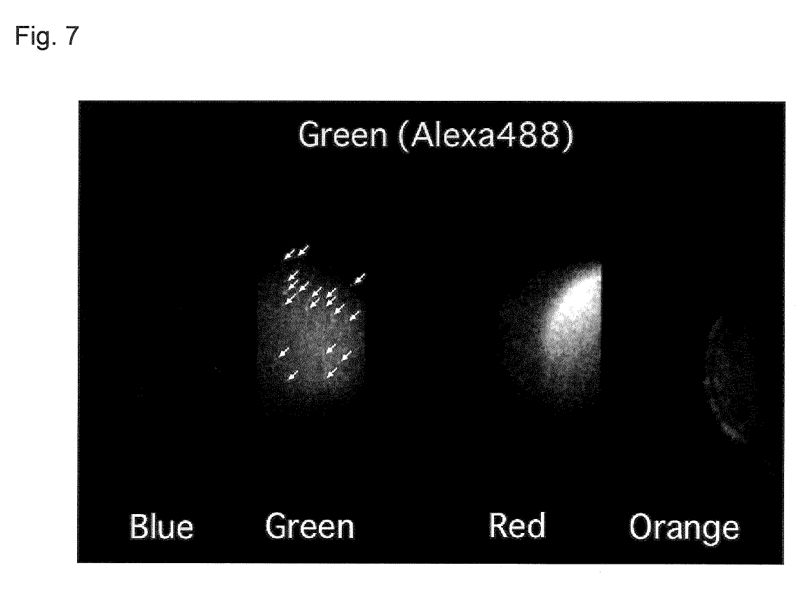

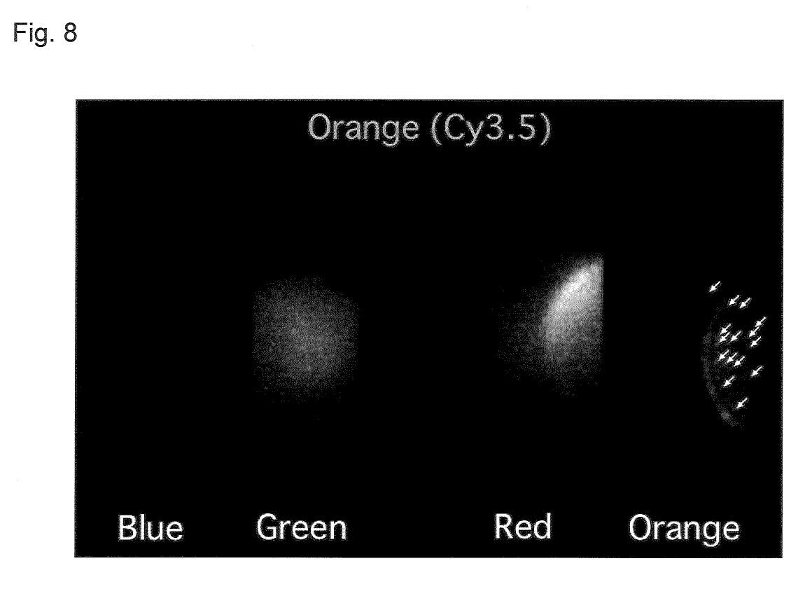

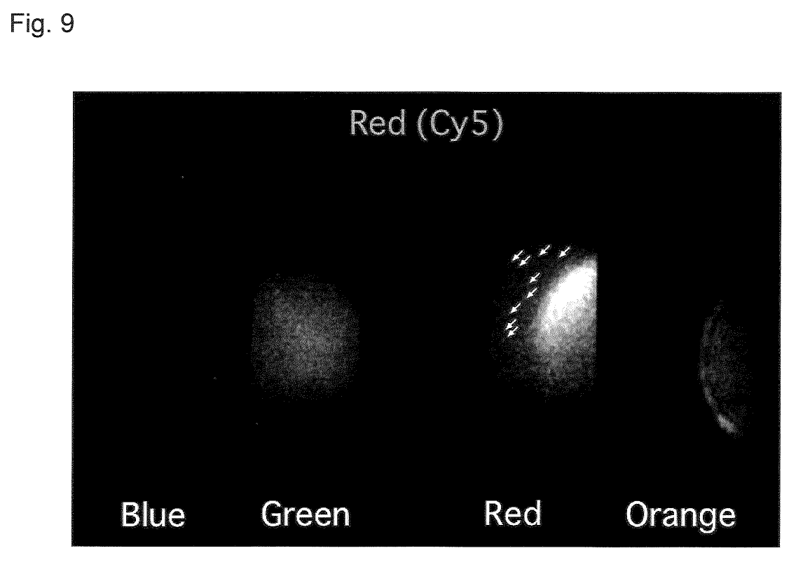

As a result of observing the respective fluorescent dyes orderly, single molecule fluorescence images were detected only in one screen image corresponding to the fluorescent dye observed (in FIGS. 6 to 9, the single-molecule fluorescence images of the fluorescent dyes were marked with white arrows). It is to be noted that the four screen images shown in each of FIGS. 6 to 9 are screen images detected in real time. FIG. 6 demonstrates that only blue fluorescence derived from Coumarine can be detected, FIG. 7 demonstrates that only green fluorescence derived from Alexa 488 can be detected, FIG. 8 demonstrates that only orange fluorescence derived from Cy3.5 can be detected, and FIG. 9 demonstrates that only red fluorescence derived from Cy5 can be detected.

### <3. Single Molecule Sequencing>

### Example 3

Sequencing was carried out using a nucleic acid having a sequence in which ten bases were continuously arranged in such a manner that adenine (A) and guanine (G) were alternately arranged (5′-GAGAG AGAGA CCCTC ACGCT GCCAT CCTCC-3′; SEQ ID No. 3) as template DNA, a biotin-labeled primer oligonucleotide (5′ Biotin-GGAGG ATGGC AGCGT GAGGG-3′; SEQ ID No. 4), and Cy3.5-labeled dCTP (Cy3.5-dCTP) and Cy5-labeled dUTP (Cy5-dUTP) as two kinds of substrates.

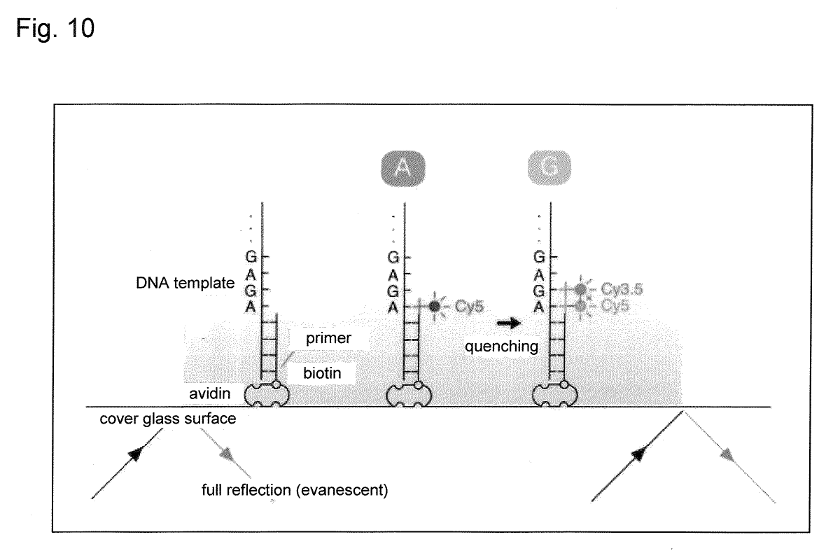

As shown in FIG. 10, the template DNA is labeled with biotin using the primer sequence, and is immobilized onto a cover glass by binding the biotin as a label to the cover glass coated with avidin. In this state, a mixed solution (10 μL) containing Cy3.5-dCTP, Cy5-dUTP, and DNA polymerase β (final concentrations: 10 μM DNA polymerase β, 0.5 nM Cy3.5-dCTP, 0.5 nM Cy5-dUTP, 12 mM Tris-HCl (pH 8.0), 0.25 mM DTT, 1 mM magnesium chloride) was introduced to initiate a DNA synthesis reaction, and then fluorescence detection was carried out. It is to be noted that the reaction efficiency of the DNA polymerase β was reduced by carrying out the reaction at room temperature and setting the concentration of the fluorescently-labeled deoxyribonucleotide to 0.5 nM so that the DNA synthesis reaction was carried out at such a slow rate that the reaction could be observed in video frames.

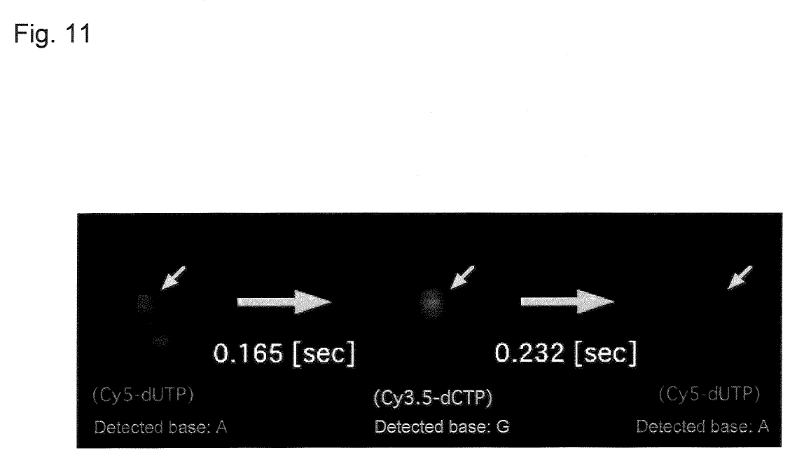

FIG. 11 shows an observational result of the real-time fluorescence detection. In order to make incorporated fluorescently-labeled deoxyribonucleotides clearly understandable, the figure was represented as images in which obtained monochrome screen images and spurious colors according to their fluorescence wavelength were superposed together. The spurious color representing Cy5-dUTP was red, and the false color representing Cy3.5-dCTP was green. As shown in FIG. 11, red fluorescence and green fluorescence were alternately detected with the lapse of time (i.e., red (Cy5-dUTP), green (Cy3.5-dCTP), red (Cy5-dUTP) . . . ). This indicates that the sequence of the template DNA is A, G, A . . . . Namely, this result demonstrates that sequencing was achieved.

In the above-described Examples, concrete forms in the scope of the present invention have been shown, however, the present invention may be practiced in various other forms without limited to these Examples. Therefore, the above-described Examples are merely exemplification in all respects, and should not be interpreted in a limitative manner. Further, any changes that belong to equivalents of claims are within the scope of the present invention.

1. A method for synthesizing a nucleic acid, including the steps of:
forming a complex of a target nucleic acid hybridized to a primer oligonucleotide and a DNA polymerase β;
allowing the DNA polymerase β to incorporate a fluorescently-labeled deoxyribonucleotide so that the fluorescently-labeled deoxyribonucleotide is bound to the 3′ end of the primer oligonucleotide; and
allowing the DNA polymerase β to continuously incorporate fluorescently-labeled deoxyribonucleotides to extend a nucleic acid complementary to the target nucleic acid from the 3′ end of the bound fluorescently-labeled deoxyribonucleotide.

2. The nucleic acid synthesis method according to claim 1, wherein the fluorescently-labeled deoxyribonucleotide is a deoxyribonucleotide labeled with an anionic fluorescent dye.

3. The nucleic acid synthesis method according to claim 2, wherein the anionic fluorescent dye is selected from a group consisting of Alexa Fluor®488, Alexa Fluor®532, Alexa Fluor®546, fluorescein, Oregon Green®488, Cy3.5, Cy5, Cy5.5, and naphthofluorescein.

4. A method for sequencing a single nucleic acid molecule, including the steps of:
forming a complex of a target nucleic acid to be sequenced hybridized to a primer oligonucleotide and a DNA polymerase β;
allowing the DNA polymerase β to incorporate a fluorescently-labeled deoxyribonucleotide so that the fluorescently-labeled deoxyribonucleotide is bound to the 3′ end of the primer oligonucleotide; and
allowing the DNA polymerase β to continuously incorporate fluorescently-labeled deoxyribonucleotides to extend a nucleic acid complementary to the target nucleic acid to be sequenced from the 3′ end of the bound fluorescently-labeled deoxyribonucleotide, wherein
fluorescence emitted from each of the fluorescently-labeled deoxyribonucleotides incorporated into the DNA polymerase β is sequentially detected to carry out the sequencing of the target nucleic acid.

5. The method for sequencing a single nucleic acid molecule according to claim 4, wherein two or more kinds of the fluorescently-labeled deoxyribonucleotides are prepared and the two or more kinds of fluorescently-labeled deoxyribonucleotides have different fluorescent labels depending on the kind of their base.

6. The method for sequencing a single nucleic acid molecule according to claim 4, wherein either the target nucleic acid to be sequenced or the DNA polymerase β is immobilized onto a substrate, and wherein an evanescent field is generated at the surface of the substrate, onto which the target nucleic acid to be sequenced or the DNA polymerase β has been immobilized, and wherein when the fluorescently-labeled deoxyribonucleotide is incorporated into the DNA polymerase β, fluorescence emitted from the incorporated fluorescently-labeled deoxyribonucleotide and excited by the evanescent field is detected.

7. The method for sequencing a single nucleic acid molecule according to any one of claim 4, wherein the fluorescently-labeled deoxyribonucleotide is a deoxyribonucleotide labeled with an anionic fluorescent dye.

8. The method for sequencing a single nucleic acid molecule according to claim 7, wherein the anionic fluorescent dye is selected from a group consisting of Alexa Fluor®488, Alexa Fluor®532, Alexa Fluor®546, fluorescein, Oregon Green®488, Cy3.5, Cy5, Cy5.5, and naphthofluorescein.


SEQUENCE LISTING (4 sequences)

SEQ ID NO: 1 - DNA, 30 bp, Artificial
  synthetic oligonucleotide
aaaaaaaaaaccctcacgctgccatcctcc

SEQ ID NO: 2 - DNA, 20 bp, Artificial
  primer
ggaggatggcagcgtgaggg

SEQ ID NO: 3 - DNA, 30 bp, Artificial
  synthetic oligonucleotide
gagagagagaccctcacgctgccatcctcc

SEQ ID NO: 4 - DNA, 20 bp, Artificial
  primer
ggaggatggcagcgtgaggg